(19) United States
(12) Patent Application Publication
(10) Pub. No.: US 2014/0276902 A1
(43) Pub. Date: Sep. 18, 2014

(54) MEDICAL INSTRUMENT FOR DISLODGEMENT, CAPTURE, AND WITHDRAWAL OF A FOREIGN BODY FROM THE VASCULATURE OF A PATIENT

(71) Applicant: Jiyong Ahn, Totawa, NJ (US)

(72) Inventor: Jiyong Ahn, Totawa, NJ (US)

(21) Appl. No.: 13/795,585

(22) Filed: Mar. 12, 2013

(51) Int. Cl. A61B 17/50

(52) U.S. Cl. CPC A61B 17/50
USPC 606/108

(57) ABSTRACT

A medical instrument for dislodging, capturing, and retrieving a foreign body from a wall of a vessel includes a vascular snare having a plurality of loops extending from a snare shaft and a grapple member.

### BACKGROUND OF THE INVENTION

1. Field of the Invention

This invention relates to medical instrument for the removal of foreign body from the vasculature of patient. In particular, the invention relates to a medical instrument including a snare and grapple member for the dislodgement, capture, and withdrawal of a foreign body from the vasculature of a patient.

2. Description of the Related Art

As disclosed in U.S. Pat. No. 6,458,145, to Ravenscroft et al, snares are known for the retrieval and removal, of foreign bodies from the vasculature of patient. However, devices such as those disclosed by Ravenscroft offer little help when the foreign body is embedded or otherwise engaged within a vessel of the vasculature system. The closed nature of the loops making up the snare makes it virtually impossible to dislodge a foreign body when resistance to its removal is encountered.

With the foregoing in mind, the present medical instrument has been developed. The medical instrument allows for the capture of a foreign body regardless of whether the foreign body is free for movement or embedded within the wall of a vessel.

### SUMMARY OF THE INVENTION

It is, therefore, an object of the present invention to provide a medical instrument for dislodging, capturing and retrieving a foreign body from a lumen or wall of a vessel. The medical instrument includes a vascular snare including a plurality of loops extending from a snare shaft and a grapple member.

It is also an object of the present invention to provide a medical instrument wherein the plurality of loops of formed from a shape memory material.

It is another object of the present invention to provide a medical instrument wherein each of the plurality of loops includes a proximal end and a distal end, wherein the proximal ends are secured to the snare shaft, and a short segment of adjacent loops are conjoined adjacent their proximal ends.

It is a further object of the present invention to provide a medical instrument wherein the snare shaft includes an open passageway in which the grapple member is positioned.

It is also an object of the present invention to provide a medical instrument wherein the grapple member includes a distal end and a proximal end, the distal end being provided with a hook element.

It is another object of the present invention to provide a medical instrument wherein an elongated grapple shaft extends from the hook element to the proximal end of the grapple member.

It is a further object of the present invention to provide a medical instrument wherein the hook element includes first end coupled directly to the grapple shaft and the second end is free of attachment, and as the hook element extends from the first end thereof to the second end thereof it defines an arcuate path lying within the same plane.

It is also an object of the present invention to provide a medical instrument including a guide catheter in which the vascular snare and the grapple member are positioned for deployment.

It is another object of the present invention to provide a medical instrument wherein the grapple member is fixedly secured to the vascular snare.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of the present medical instrument with the grapple member extending through the vascular snare.

FIG. 2 is an exploded perspective view of the present medical instrument.

FIGS. 3 and 4 are different embodiment of loop constructions that may be employed in accordance with the present invention.

FIG. 5 is a detailed cross sectional, view showing grapple member extending through the snare shaft.

FIGS. 6, 7, and 8 show various embodiments for the shape of hook element employed in accordance with the present invention.

FIGS. 9 to 13 show steps in an exemplary usage of the present invention.

FIG. 14 is a side view of the present medical instrument engaging a foreign object.

FIG. 15 shows an alternate embodiment of the present medical instrument.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

Referring to FIGS. 1 to 14, a medical instrument 10 providing, in combination, a vascular snare 12 and a grapple member 14, is disclosed. The combination of elements provided by the present medical instrument 10 allows for the dislodgement of foreign bodies 16 from the lumen 17 and/or wall 18 of a vessel 20 and subsequent removal of the foreign body 16.

Considering first the vascular snare 12, the vascular snare 12 includes a plurality of adjacent elongated loops 22, 24, 26, 28 formed from a shape memory material. As will be appreciated based upon the following disclosure, the loops 22, 24, 26, 28 are shaped and dimensioned to expand to a desired configuration for retrieval of foreign bodies 16 from the vascular system of a patient. Each of the loops 22, 24, 26, 28 includes a proximal end 30 and a distal end 32. The distal end 32 of each of the loops 22, 24, 26, 28 is free and not interconnected with adjacent loops. Each side of each of the loops 22, 24, 26, 28 is directly interconnected with a side of an adjacent loop at a position adjacent the proximal end 30 of the loop. The vascular snare 12 also includes a flexible snare shaft 60 connected to the proximal ends 30 of the loops 22, 24, 26, 28, wherein the proximal ends 30 of the loops 22, 24, 26, 28 are gathered together at the snare shaft 60. The vascular snare 12 is further provided with a grapple member 14 shaped and dimensioned for use in dislodging embedded components, for example of a catheter, from the vascular wall 18.

In accordance with a preferred embodiment, the vascular snare 12 includes a distal end 82 and a proximal end 84. The plurality of loops 22, 24, 26, 28 are located at the distal end 82 of the vascular snare 12 and a handle 86 is provided at the proximal end 84 of the vascular snare 12. A flexible snare shaft 60 extends between the proximal end 84 and the distal end 82 of the vascular snare 12.

The first, second, third and fourth loops 22, 24, 26, 28 are respectively composed of first, second, third and fourth wires 34, 36, 38, 40. Each of the first, second, third and fourth wires 34, 36, 38, 40 are formed preferably of a multi-strand composite. As with the snare disclosed in U.S. Pat. No. 6,458,145, entitled “INTRA VASCULAR SNARE AND METHOD FOR FORMING THE SAME”, which to the extent relevant is incorporated herein, this composite is preferably constructed of nitinol and a noble metal such as gold or platinum iridium, so that the noble metal provides radiopacity while the nitinol provides shape memory. It is appreciated, the multiple strands forming the first, second, third and fourth wires 34, 36, 38, 40 can be braided, knitted, woven or wound. Other materials could be used to form the wires such as beta-titanium, MP35N, stainless steel, and possibly fibers such as Dacron (polyester) or Kevlar.

To interlace the multi-strand first, second, third and fourth wires 34, 36, 38, 40, the wires are braided adjacent their respective free ends 52, 54, 56, 58. In this way it is possible to create four loops 22, 24, 26, 28 with the conjoined short segments 88a, 88b, 90a, 90b, 92a, 92b, 94a, 94b linking adjacent free ends 52, 54, 56, 58 of the wires 34, 36, 38, 40. In particular, a free end 52 of the first wire 34 is braided with a free end 54 of the second wire 36, the other free end 54 of the second wire 36 is braided with a free end 56 of the third wire 38, the other free end 56 of the third wire 38 is braided with a free end 58 of the fourth wire 40 and the free end 52 of the fourth wire 40 is braided with the other free end 52 of the first wire 34.

While braiding is contemplated in accordance with a preferred embodiment of the present invention, the procedure for securing adjacent wires as described in the '145 patent may be employed. In accordance with such a procedure, a fine gauge hypodermic needle is used to penetrate between the strands of the first wire so that approximately fifty percent of the strands forming the first wire are on each side of the needle. Then the second wire is passed through the hypodermic needle and the first wire, and the hypodermic needle is withdrawn. This creates an interlaced joinder point between the first wire and the second wire. The procedure is then continued until all of the wires are connected in the manner disclosed herein. Once the four wires are interlaced, they form a square, the size of which dictates the ultimate diameter of the intravascular snare. The portions of the first, second, third and fourth wires which form the four sides of the square are then moved outwardly to form the free distal ends of first, second, third and fourth loops.

It should be recognized that although four loops formed from four wires are preferred for the intravascular snare of the present invention, the snare could be formed from two interconnected wires, three interconnected wires or from more than four wires.

To complete the formation of the first, second, third and fourth loops 22, 24, 26, 28, the free ends 52 of the first wire 34, the free ends 54 of the second wire 36, the free ends 56 of the third wire 38 and the free ends 58 of the fourth wire 40, which extend outwardly beyond the conjoined short segments 88a, 88b, 90a, 90b, 92a, 92b, 94a, 94b are gathered sequentially to a central point which is attached to the snare shaft 60. The snare shaft 60 provides an open passageway 62 for the extension of the grapple member 14.

In accordance with a preferred embodiment, the free distal ends 32 of the loops 22, 24, 26, 28 are left free to engage the foreign object to be removed from a vessel 20. In addition to the braiding of the wires during the formation of the first, second, third and fourth loops 22, 24, 26, 28, the loops are joined to adjacent loops along limited segments, via the braiding discussed above, in the area adjacent the proximal ends 30 of the loops 22, 24, 26, 28. In particular, a short segment 88a of the first loop 22 adjacent the proximal end 30 is conjoined with a short segment 88b of similar length of the second loop 24 adjacent the proximal end 30, a short segment 90a of the second loop 24 adjacent the proximal end 30 is conjoined with a short segment 90h of similar length of the third loop 26 adjacent the proximal end 30, a short segment 92a of the third loop 26 adjacent the proximal end 30 is conjoined with a short segment 92b of similar length of the fourth loop 28 adjacent the proximal end 30, and a short segment 94a of the fourth loop 28 adjacent the proximal end 30 is conjoined with a short segment 94b of similar length of the first loop 22 adjacent the proximal end 30.

Once the first, second, third and fourth loops 22, 24, 26, 28 are formed, the vascular snare 12 is positioned on a forming jig to form the geometry of the loops. The loops 22, 24, 26, 28 are formed to extend from the snare shaft 60 at angles of approximately fifteen degrees, and spaced from the end of the snare shaft 60, each loop 22, 24, 26, 28 flares outwardly for approximately another fifteen degrees to open the distal ends 32 of the loops 22, 24, 26, 28 to a greater diameter that ultimately increases wire to vessel surface area contact. The loops can be formed to other combined angles which will provide loops which extend up to forty degrees with respect to the shaft. In accordance with a preferred embodiment, either circular loops (see FIG. 3) or elongated loops (see FIG. 4) are preferred. It is appreciated the circular loops as shown in FIG. 3 will function best in straight vessel applications and the elongated loops shown in FIG. 4 will function best in situations where angular orientations are likely to be confronted.

To further enhance radiopacity, the loops can be coated with noble metals by dip casting or ion disposition. A preferred method is by an unbalanced magnetron sputtering process providing a well-adhered noble coating on the outside surfaces of the braid. This will prevent coating fracture during loop deformation. Radiopacity can also be achieved without a coating process by fabricating one or more strands of the multi-strand material forming the loop from a radiopaque material such as a noble metal or from a tubular strand filled with a radiopaque material. In some instances, the first, second, third and fourth wires 34, 36, 38, 40 may be formed from a single strand of wire filled with a radiopaque material.

The vascular snare 12 is preferably formed of nitinol wire or similar thermally responsive shape memory material, and may then be cooled to the martensitie state for insertion into a catheter or delivery tube. When the vascular snare 12 is projected outwardly from the delivery tube within a body vessel 20, it can be formed to return to the austenitic state in response to body temperature and expand outwardly into contact with the vessel 20 walls.

By shaping the snare in accordance with known techniques, the material from which the snare if composed can have one shape remembered in the austenitic phase if elastically cycled and a second shape can be formed by controlled deformation in the martensite phase. Once the new shape is formed, the material will remember the new shape if cycled elastically. Thus a medical device in the martensitic state can be crushed within a delivery device to alter the remembered austenitic shape of the medical device.

in addition to the vascular snare 12, the present medical instrument 10 is provided with a grapple member 14. The grapple member 14 includes a distal end 64 and a proximal end 66. The distal end 64 is provided with a hook element 68 with an elongated grapple shaft 70 extending therefrom and to the proximal end 66 of the grapple member 14. The grapple shaft 70 is flexible and is shaped and dimensioned to fit within the open passageway 62 of the snare shaft 60 for movement relative thereto. The grapple member 14 is provided with a handle structure 74 at the proximal end 66 thereof providing a medical practitioner with control over the movement of the hook element 68 at the distal end 64 of the grapple member 14. As such, the grapple member 14 is shaped for movement within the snare shaft 60 in such a manner that the grapple member 14 may be selectively removed from and positioned within the snare shaft as required.

With the foregoing overview of the structure of the grapple member 14 in mind, the hook element 68 includes a fixed first end 96 and a free second end 98. The body of the hook element 68 as it extends from the first end 96 to the second end 98 follows an arcuate path lying within the same plane such that the free second end 98 extends in a direction oblique to the longitudinal axis in which the hook element 68 extends adjacent to the fixed first end 96. The second end 98 is further provided with non-traumatic features enhancing its performance in a manner that will be appreciated based upon the following disclosure discussing the use of the present medical instrument 10. For example, the free second end 98 includes a small diameter loop 100 at its very end which functions to enhance the nontraumatic characteristics of the grapple member 14.

It is appreciated, the hook element may be formed with other shapes, for example, the hook element may be formed with a planar arcuate structure extending about an arc of approximately 270 degrees (see FIGS. 1, 2 and 5), a planar arcuate structure with an angled attachment at the first end 96 and an arcuate body of approximately 180 degrees such that the tip at the second end 98 is in substantial alignment with the longitudinal axis of the grapple shaft 70 (see FIG. 6), an arcuate structure with an angled attachment at the first end 96 and an arcuate body of only approximately 90 degrees such that the tip at the second end 98 extends in a direction substantial in alignment with the longitudinal axis of the grapple shaft 70 (see FIG. 7), or a helical shaped hook (see FIG. 8). It is appreciated each of these shapes offer advantages for specific anatomies and specific applications.

As with the vascular snare 12, the hook element 68 is preferably formed of nitinol wire or similar thermally responsive shape memory material, and may then be cooled to the martensitic state for insertion into a catheter or delivery tube. As such, the hook element 68 is substantially straight prior to deployment. When the hook element 68 is projected outwardly from the delivery tube 76 and the vascular snare 12 within a body vessel 20, it takes a desired hook shape (that is, for example, the hook shape as shown in FIGS. 1, 2, 5-8, 14 and 15) in response to body temperature. In addition, and as with the vascular snare 12, the hook element 68 may be coated to enhance radiopacity.

The vascular snare 12 and the grapple member 14 are housed within a guide, or delivery, catheter 76. This allows for relative movement of both the vascular snare 12 and the grapple member 14 within the guide catheter 76. In an effort enhance visualization of the guide catheter 76, the distal end 78 of the guide catheter 76 is provided with radiopaque markings 80.

In accordance with the use of the present instrument, and with reference to FIGS. 9-13, a foreign body 16 is identified in the vascular system of a patient. The guide catheter 76 is thereafter deployed within the vascular system with its distal end. 78 adjacent the foreign body 16 requiring retrieval. The vascular snare 12 and grapple member 14 are then pass through the guide catheter 76 until the first, second, third and fourth loops 22, 24, 26, 28, as well as the grapple member 14, are fully extended from the distal end 78 of the guide catheter 76, allowing the loops 22, 24, 26, 28 to take their desired configuration.

With the grapple member 14 and the first, second, third and fourth loops 22, 24, 26, 28 fully deployed, the hook element 68 of the grapple member 14 is utilized in disengaging the foreign body 16 from engagement with the vessel 20 wall 18. Once the foreign body 16 is fully disengaged from the vessel 20 wall 18, the loops 22, 24, 26, 28 and the hook element 68 are engaged with the foreign body 16. With the foreign body 16 fully engaged with the vascular snare 12 and the grapple member 14, both the loops 22, 24, 26, 28 and the grapple member 14 are drawn into the guide catheter 76 along the foreign body 16. The vascular snare 12, grapple member 14 and the foreign body 16 may then be fully withdrawn from the guide catheter 76 and the patient.

With reference to FIG. 15, an alternate embodiment of the present invention is disclosed. In accordance such an embodiment, medical instrument 110 includes a grapple member 114 that is fixedly secured to the vascular snare 112. The vascular snare 112 is substantially as described above, but the snare shaft 112 is constructed without an open passageway. As such, the grapple member 114 is composed of a hook element 168 with a short grapple shaft 170 secured at the juncture of the loops 122 and the snare shaft 160. The hook element 168 and grapple shaft 170, therefore, extend from the distal end 182 of the snare shaft 160, preferably, with the grapple shaft 170 in alignment with the longitudinal axis of the snare shaft 160.

While the preferred embodiments have been shown and described, it will be understood that there is no intent to limit the invention by such disclosure, but rather, is intended to cover all modifications and alternate constructions falling within the spirit and scope of the invention.

1. (canceled)

2. The medical instrument according to claim 10, wherein the plurality of loops of formed from a shape memory material.

3-6. (canceled)

7. The medical instrument according to claim 10, wherein as the hook element extends from the first end thereof to the second end thereof it defines an arcuate path lying within the same plane.

8. The medical instrument according to claim 10, further including a guide catheter in which the vascular snare and the grapple member are positioned for deployment.

9. The medical instrument according to claim 10, wherein the grapple member is fixedly secured to the vascular snare.

10. A medical instrument for dislodging, capturing and retrieving a foreign body from a lumen or wall of a vessel, comprising:
a vascular snare includes a snare shaft including an open passageway and a plurality of loops extending from the snare shaft, each of the plurality of loops includes a proximal end and a distal end, wherein the proximal ends are secured to the snare shaft;
a grapple member is positioned in the snare shaft,
wherein each of the plurality of loops includes a short segment, and the short segment of adjacent loops are conjoined adjacent the proximal ends of each of the plurality of loops and the distal ends of the each of the plurality of loops are connected to the short segment and compose a loop shape;
the grapple member includes a proximal end and a distal end, the distal end being provided with a hook element, and an elongated grapple shaft extends from the hook element to the proximal end of the grapple member; and
the hook element includes a first end coupled directly to the grapple shaft and the second end is free of attachment.
